(19) United States
(12) Patent Application Publication
(10) Pub. No.: US 2009/0062914 A1
(43) Pub. Date: Mar. 5, 2009

(54) DEVICES AND METHODS FOR INTERVERTEBRAL THERAPY

(76) Inventor: James F. Marino, La Jolla, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/200,830

(22) Filed: Aug. 28, 2008

Related U.S. Application Data
(60) Provisional application No. 60/966,851, filed on Aug. 29, 2007.

(51) Int. Cl. A61F 2/02; A61B 19/00; A61B 17/04

(52) U.S. Cl. 623/11.11; 128/898; 606/301

(57) ABSTRACT

Disclosed are devices, systems and methods for treating vertebral diseases and injuries, and more particularly to devices, systems and methods of delivering therapy to the spine within or in close proximity to the intervertebral disc space or vertebral endplate. The method involves positioning an implant having an internal passageway with a proximal and distal opening into a vertebra such that the distal opening is adjacent a delivery site such as an intervertebral disc space or a vertebral endplate. The method also involves delivery a therapeutic substance through the proximal opening and through the internal passageway to the delivery site while the implant is positioned in the vertebra. The devices, systems and methods can be performed with and without spinal fixation.

### REFERENCE TO PRIORITY DOCUMENT

This application claims the benefit of priority of co-pending U.S. provisional patent application Ser. No. 60/966,851, entitled “Devices and Methods for Intervertebral Therapy”, filed Aug. 29, 2007. Priority of the aforementioned filing date is hereby claimed and the entire disclosure of which is hereby incorporated by reference.

### BACKGROUND

The surgical management and correction of spinal impairments or deformities such as idiopathic scoliosis has historically involved the use of complicated interventions and implantation of rigid fixation device systems. The spinal implant systems tend to be elaborate and require extensive skill, time and instrumentation to implant that can lead to increased morbidity for the patient and increased operating expense.

### SUMMARY

In view of the foregoing, there is a need for improved treatments for spinal impairments and deformities. A need also exists for an improved spinal fixation system that is capable of accepting therapeutic agents before, during, and/or after surgical implantation, holding those agents, and also providing in vivo delivery of those agents to the surrounding tissues including intervertebral disc space and the vertebral endplates. Furthermore, a need exists for a spinal drug delivery system that can be repeatedly replenished with therapeutic agents, and that can accept a wide range of therapeutic agents. Disclosed herein are devices, systems and methods for treating spinal impairments and deformities.

One embodiment of a bone therapy method includes providing an implant having an internal passageway with a proximal opening and a distal opening. The implant is positioned into a vertebra such that the distal opening is adjacent a delivery site, such as an intervertebral disc space or a vertebral endplate. A therapeutic substance is delivered through the proximal opening and through the internal passageway to the delivery site while the implant is positioned in the vertebra.

Also disclosed is a method of treating a patient having idiopathic adolescent scoliosis. The method includes providing a first implant and second implant each having an internal passageway with a proximal opening and a distal opening. The method also includes positioning the first implant into a vertebra on a concave side of the patient's spine such that the distal opening of the first implant is adjacent a first delivery site. The method also includes positioning the second implant into a vertebra on a convex side of the patient's spine such that the distal opening of the second implant is adjacent a second delivery site. The method also includes delivering a growth-stimulating agent to the first delivery site through the first implant to stimulate growth on the concave side of the spine; and delivering a growth-inhibitory agent to the second delivery site through the second implant to retard growth on the convex side of the spine.

The details of one or more embodiments are set forth in the accompanying drawings and the description below. Other features, objects, and advantages will be apparent from the description and drawings, and from the claims.

### DESCRIPTION OF DRAWINGS

FIG. 1 shows an exemplary view of a vertebral column.

FIG. 2 shows a superior view of a vertebra.

FIG. 3 shows a lateral view of a vertebra.

FIG. 4 shows an exemplary embodiment of a monoplanar polyaxial fixation system in an assembled state.

FIG. 5 shows the fixation system of FIG. 4 in an exploded state.

FIG. 6 shows an example of a fixation system implanted in a spine.

FIGS. 7A and 7B show cross-sectional side views of embodiments of a fixation and drug delivery system.

FIGS. 8A and 8B show cross-sectional side views of embodiments of a drug delivery system.

FIG. 9A shows a schematic view of another embodiment of a fixation and drug delivery system.

FIG. 9B shows a schematic view of another embodiment of a drug delivery system.

FIGS. 10A-10C show a cross-section view of vertebrae illustrating exemplary orientations of drug delivery systems.

### DETAILED DESCRIPTION

Disclosed are devices, systems and methods for treating vertebral diseases and injuries, and more particularly to devices, systems and methods of delivering therapy to the spine within or in close proximity to the intervertebral disc space or vertebral endplate. The devices, systems and methods can be performed with and without spinal fixation.

Bone stabilization assemblies are commonly used throughout the skeletal system to stabilize broken, fractured, diseased or deformed bones. For example, screw systems can be adapted for the fixation and manipulation of the bones of the vertebral column. Screw systems can be used to correct deformity, and/or to treat trauma. They can be used in instrumentation procedures to affix rods and plates to the spine. They can also be used to immobilize part of the spine to assist fusion by holding bony structures together. We describe herein the use of fixation systems that can be used as drug delivery systems.

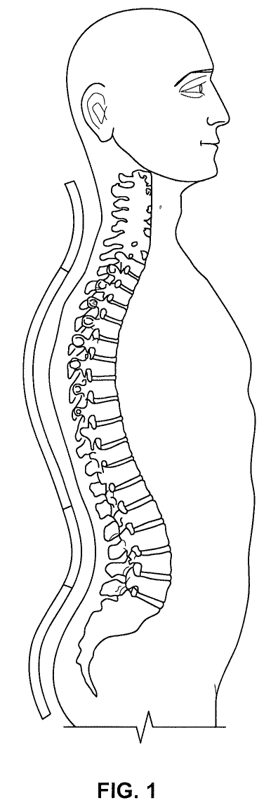

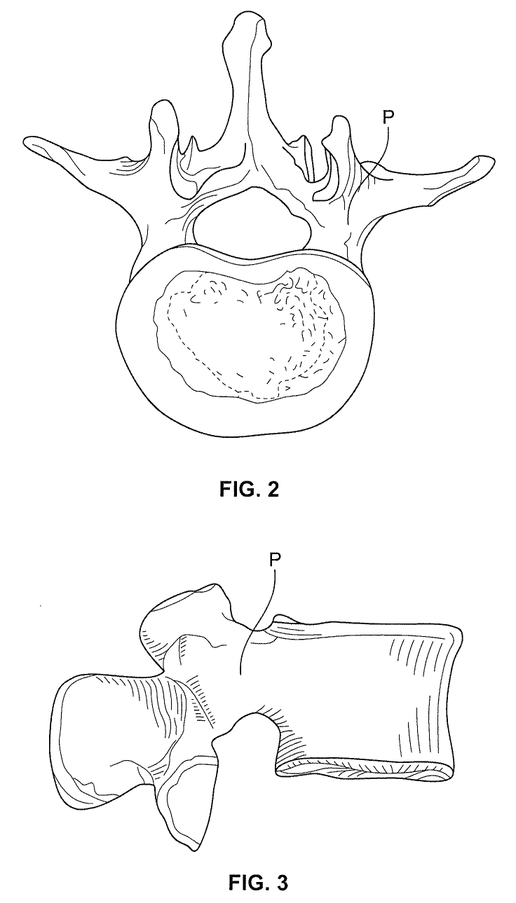

A vertebral pedicle is a dense stem-like structure that projects from the posterior of a vertebra. There are two pedicles per vertebra that connect to other structures such as the lamina and vertebral arch. FIGS. 1, 2 and 3 show exemplary views of a typical vertebral column, a superior view of a typical vertebra, and a lateral view of a typical vertebra, respectively.

A pedicle screw is a particular type of bone screw designed for vertebral fixation upon implantation into a vertebral pedicle. A typical pedicle fixation system includes a screw having a shank and a head. The screw removably couples to a tulip-like coupling element that can also be coupled to a fixation rod. One type of pedicle fixation system is a monoaxial pedicle screw system in which the axis of the screw shank is fixed relative to the coupling element. Another more common type of pedicle fixation system is a polyaxial system in which the axis of the screw can be varied through different planes relative to the coupling element. In another type of polyaxial system, the axis of the receiving element can be varied relative to the screw shaft but only within a single plane.

The methods described herein are described in the context of use with such a single-plane polyaxial pedicle implant system. However, it should be appreciated that the disclosed methods can be used with any type of pedicle implant system. It should also be appreciated that the disclosed methods can be used with any implant device, not solely bone screws or fixation systems. Thus, although the methods are described herein in the context of use with a bone implant having an internal passageway and it should be appreciated that the methods are not limited to use with a bone screw.

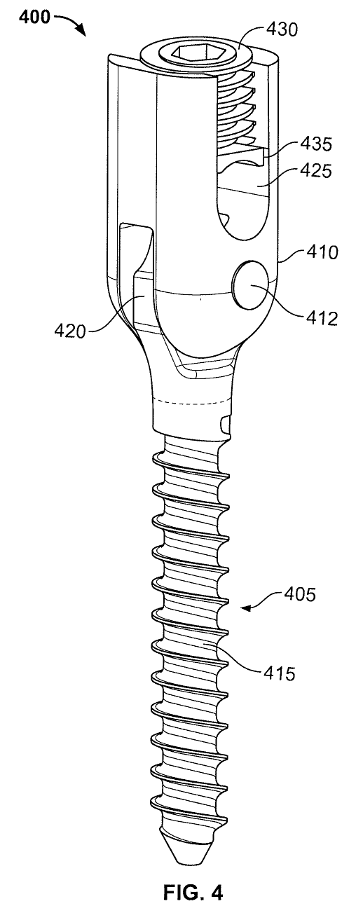

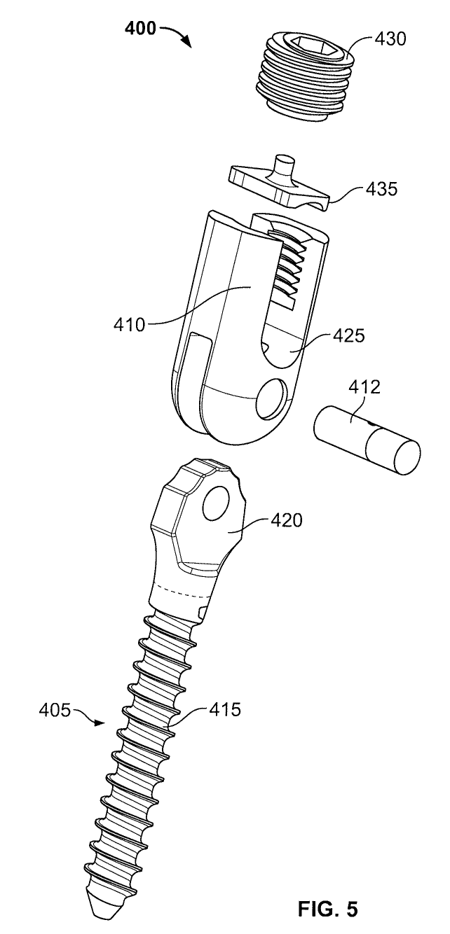

FIG. 4 shows an exemplary embodiment of a monoplanar, polyaxial fixation system 400 in an assembled state. FIG. 5 shows the system 400 in an exploded state. The system 400 includes an implant 405 that couples to a coupling element 410 via a pin 412 that mates with both the coupling element 410 and a head 420 of the implant 405. The implant 405 can rotate relative to the coupling element 410 about an axis defined by the pin 412. The implant 405 includes a shank 415 adapted to be fixed to bone.

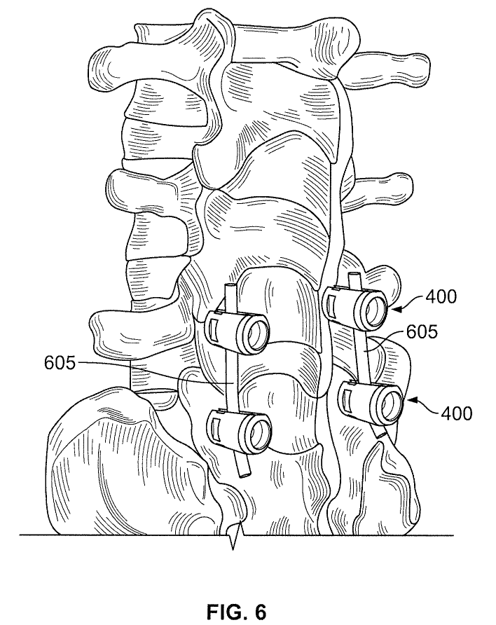

The coupling element 410 includes a u-shaped rod-receiving channel 425 for receiving a rod 605 (shown in FIG. 6). The rod 605 can be secured to the coupling element 410 via a set screw 430 that exerts a downward force onto the rod 605 when the rod 605 is positioned within the rod-receiving channel 425. An upper compression member 435 can be interposed between the set screw 430 and the rod 605 for distributing the downward force over the surface of the rod 605.

As mentioned, the fixation system 400 shown in FIGS. 4 and 5 is exemplary. The methods described herein are not limited to use with the specific pedicle system shown in the figures, but can rather be used with other types of pedicle systems or other bone implant systems.

In use, a receiving channel can be drilled into the bone and the shank 415 of the implant 405 inserted into the channel. Alternately, the shank 415 can be positioned into the bone without a channel being pre-formed. The implant 405 and coupling element 410 collectively act as a firm anchor point that can then be connected such as with a rod to another fixation system. As shown in FIG. 6, in one embodiment, the fixation systems 400 are placed down the small bony tube created by the pedicles on each side of the vertebra, between the nerve roots. This allows the implants to grab into the bone of the vertebral body, giving them a solid hold on the vertebra. Once the implants are placed, one in each of the two pedicles of each vertebra, rods 605 are then attached to the implants to connect the implants together. The implants are placed at two or more consecutive spine segments (e.g., lumbar segment 5 and 6) and connected by the rods. It should be appreciated that the implants can be positioned at other spinal locations and are not limited to being positioned through the pedicles.

In addition to fixation of broken, fractured, diseased or deformed bones, implants can be used as a conduit for the delivery of localized treatment to part(s) of the spine, including the vertebral endplate, the intervertebral disc space, ring apophysis or growth plate of the vertebral body, etc. It should be understood that implants can be used simultaneously as fixation devices and as conduits for the localized delivery of therapeutic agents. One advantage of using a threaded implant as the conduit for treatment is that it can be used with distraction or fixation elements thereby removing the load from the target region and promoting healing. It should be appreciated that a treatment conduit need not be a threaded implant. The treatment conduit can be other implantable devices, whether threaded or non-threaded, that provide a conduit for the delivery of localized treatment to part(s) of the spine.

It should be understood that an implant can be used for the sole purpose of localized delivery of agents (e.g. pharmaceuticals, growth factors, cellular elements, cell therapy, cement or other hardening materials). For example, a small diameter “drug delivery implant” can be used that provides access to targeted regions of the spine, such as the vascular bone immediately adjacent to the endplate or into the intervertebral disc space. Such a drug delivery implant could be used to place a catheter into the vertebra for delivery of therapeutic agents independent of any fixation or motion sparing constructs. It should also be appreciated that a drug delivery implant need not be threaded. Instead, a simple conduit driven into the bone can be used. For the sake of simplicity, the term “implant” will be used and can encompass all these potential embodiments of the device.

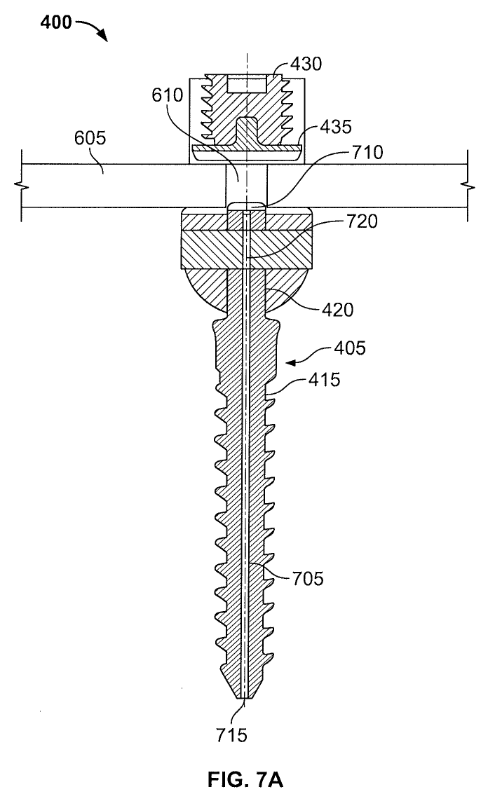

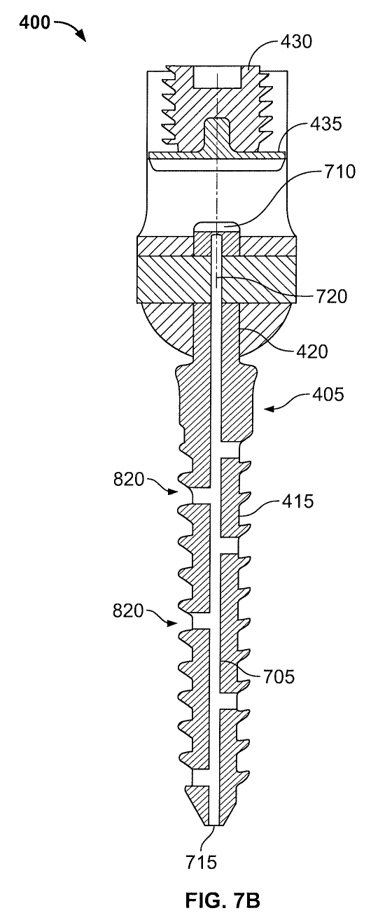

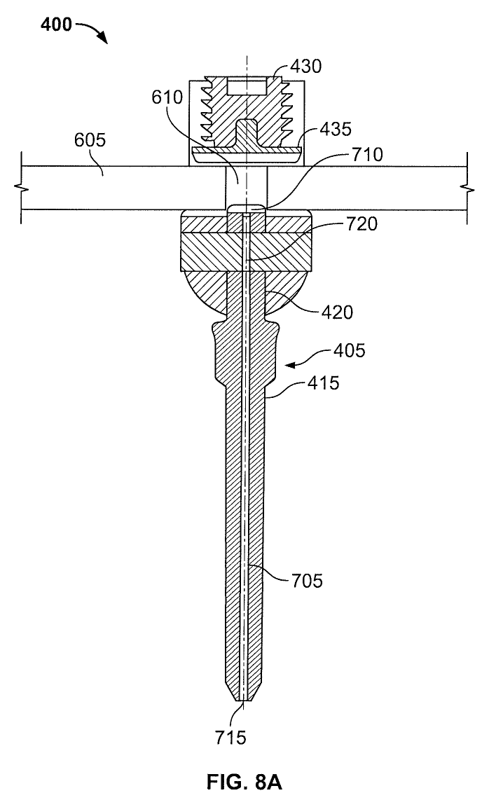

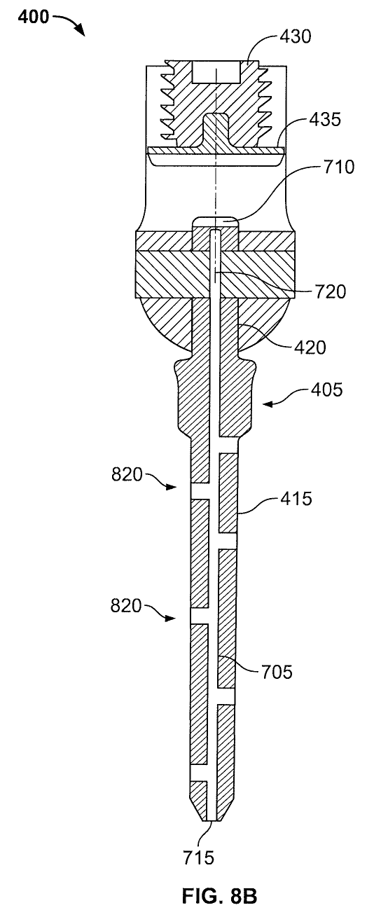

FIG. 7A shows a cross-sectional side view of a fixation and drug delivery system 400. FIG. 8A illustrates an embodiment of a drug delivery system 400 in which the shank 415 of the implant 405 is not threaded and does not provide an anchor point. The systems 400 include a passageway 705 that can be used to deliver a material, such as a drug, therapeutic substance, or device, to the bone or to surrounding tissue. In the illustrated embodiments, the shank 415 of the implant 405 includes an elongated passageway 705. The passageway 705 extends internally along the length of the shank 415 and through the implant head 420 such that a proximal opening 710 is disposed at or near an upper end of the implant 405. Likewise, a distal opening 715 is disposed at or near the lower tip of the shank 415. The passageway 705 and openings 710 and 715 collectively form a conduit through which a material and/or a device can be conveyed into the bone or surrounding tissue when the implant 405 is positioned in the spine.

With reference still to FIGS. 7A and 8A, other components of the systems 400 can also include one or more passageways that align with the passageway 705 in the shank 415. For example, the pin 412 can include a passageway 720 that aligns with the passageway 705 in the implant 405 when the system is assembled. In this manner, the pin 412 does not obstruct or otherwise impede the passageway 705 of the implant 405. It should be appreciated that any of the other components of the systems 400 can have passageways that align with the passageway 705 of the implant 405. For example, the set screw 430 and/or the compression member 435 can have passageways that align with the passageway 705.

Similarly, if a connecting rod 605 is used such as in the embodiment of the fixation and drug delivery system of FIG. 7B, such as for spinal fixation in combination with delivery of therapeutic materials, the rod 605 can also have fenestrations or passageways 610 such that passage of the material from the most proximal portion of the implant 405 can be delivered to the distal end of the implant 405. Accordingly, an uninterrupted passageway can extend through the fixation and drug delivery system 400 from the lower tip of the implant 405 to the top of the coupling element 425. Alternately, any of the components do not include a passageway such that the component serves as a cap that blocks or encloses the passageway 705 in the assembled system.

In an alternate embodiment, the shank 415 of the implant 405 can be fenestrated (see FIGS. 7B and 8B) in addition to having a passageway 705. The fenestrations 820 can connect with the passageway 705 such that material and/or a device can be conveyed into the bone tissue through the shaft 415. In an embodiment, the distal opening 715 is sealed. This prevents delivery of material through the distal end of the implant, but allows for delivery of material out the fenestrations 820 to be conveyed into the bone or surrounding tissue when the implant 405 is positioned in the spine. Such an embodiment would be useful, for example, to prevent inadvertent delivery of material into the retroperitoneal space when the implant 405 is positioned at the L5/S1 level where the distal end of the implant can protrude out of the bone.

When the implant 405 is in position, the passageway 705 serves as a conduit through which a substance, device, or other material can be delivered to the vertebra or surrounding tissue. The proximal opening 710 serves as an entryway for delivering the material into the passageway 705 while the distal opening 715 serves as an exitway for the material to communicate with the vertebra or surrounding tissue. Thus, the implant 405 is desirably positioned in the vertebra such that the opening 715 is at or near the location where material or a device is to be delivered via the passageway 705. Alternatively, fenestrations 820 (FIGS. 7B and 8B) can serve as an additional exitway for the material to communicate with the bone tissue through which the shaft 415 is positioned.

Any of a variety of materials can be delivered to the vertebra or surrounding tissue via the passageway 705. In an embodiment, the passageway 705 provides a conduit for one or more devices to access the vertebra or surrounding tissue. For example, any of a variety of devices, such as suture devices, catheters, cannulae, needles, drug delivery devices, etc. can be inserted through the passageway 705. The device to be inserted desirably is of a size that can fit through the passageway 705. In this regard, the device can be configured to transition between a reduced size that fits through the passageway and an enlarged size that is larger than the cross-sectional size of the passageway. The change in size can be achieved, for example, through the use of shape-memory material such as Nitinol. The passageway 705 can have any of a variety of cross-sectional shapes such as, but not limited to, circular, square, oval, rectangular, and irregular shapes. Moreover, the cross-sectional shape or size of the passageway can vary moving along the length of the passageway.

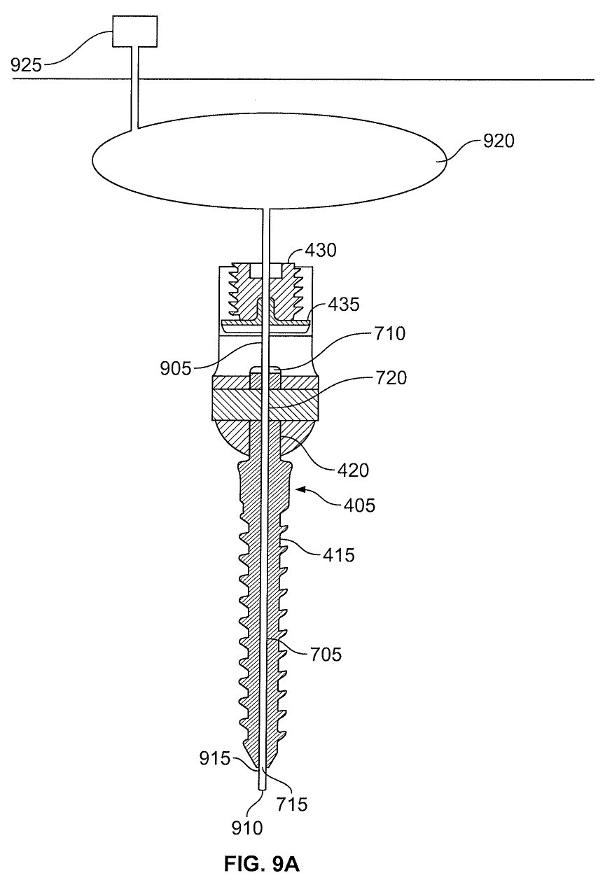

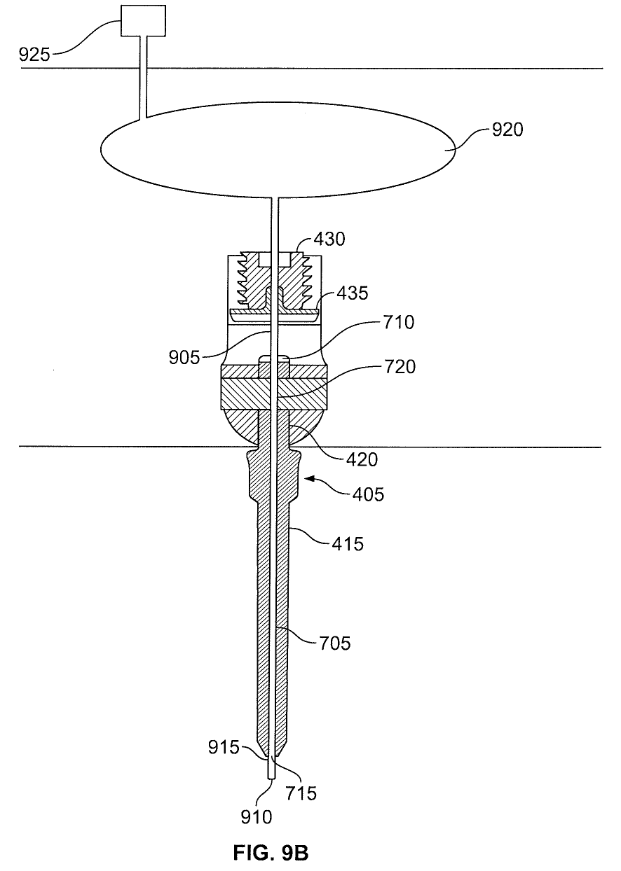

FIGS. 9A and 9B show embodiments in which a delivery catheter 905 is inserted through the passageway 705 such that a distal tip 910 of the catheter 905 protrudes out of or is positioned near the distal opening 715 of the passageway 705. The delivery catheter 905 can include an internal passageway 915 that serves as a conduit for passage of a substance. In this manner, the delivery catheter 905 can be used to deliver the substance to the vertebra or surrounding tissue via the passageway 705 in the implant 405. If the implant 405 is of the embodiment of FIG. 7B or 8B in which fenestrations 820 are present in the shaft 415, the catheter 905 would likewise have fenestrations.

The delivery catheter 905 can extend into the passageway 705 or can mechanically couple to an upper region of the implant to form a connection between a distal end of the catheter and a portion of the implant. In the case where the catheter 905 mechanically connects to the implant, there is desirably a hermetic seal between the catheter and the implant to prevent agent from leaking at the connection location.

The method and devices disclosed herein allow for a more localized delivery of agents to the desired treatment site. The agents can be delivered in stages to the bone. The localized delivery of agent permits a relatively high local and low systemic concentrations of agent, substance, factor, etc. used. The substances delivered by the system can vary. Substances can include pharmaceuticals or other therapeutic substances, such as osteoproliferative materials, osteogenic materials, chondroproliferative factors, chondrogenic factors, extracellular matrix-inducing factors, antibiotic, analgesic, anesthetic, anti-inflammatory agent, therapeutic protein, cytokine, and chemokine, cellular elements, cell therapy agents, radiographic material, osteoinductive material, antibiotics, analgesics, anesthetics, anti-inflammatory agents, therapeutic proteins, cytokines, chemokines, growth factors such as insulin like growth factor types I and II, and growth inhibitory substances like insulin-like growth factor binding protein type-1. It should be understood that more than one substance can be delivered. For example, more than one agent can be delivered concurrently to enable combination therapies. Similarly, concurrent delivery of more than one agent can be delivered to more than one location along the spine.

Still with respect to FIGS. 9A and 9B in which a catheter 905 is threaded through the passageway 705 of the system 400, the catheter 905 can be associated with a reservoir 920 near its proximal end. The reservoir 920 can be implanted, for example, under the skin or fascia, in such a way that it is externally accessible. For example, the reservoir 920 can be associated with an implanted access port 925, such as a self-sealing hypodermic needle access port. The access port 925 can be positioned under the skin or fascia at a site to allow for easy access with a needle such as a Huber needle. The site can be over a bone such as the ilium, for example, just inferior and posterior to the anterior superior iliac spine. Further, the reservoir 920 and/or access port 925 can be adjacent, within, or affixed to a bone such as the ilium. Such a configuration would be beneficial to cosmesis and patient comfort.

More than one reservoir 920 can be used to deliver concurrent therapies of more than one agent, as described in more detail below. The catheter 905 can also be connected to a pump, for example, an externally-programmable pump, used to control the rate of delivery of therapeutic substances.

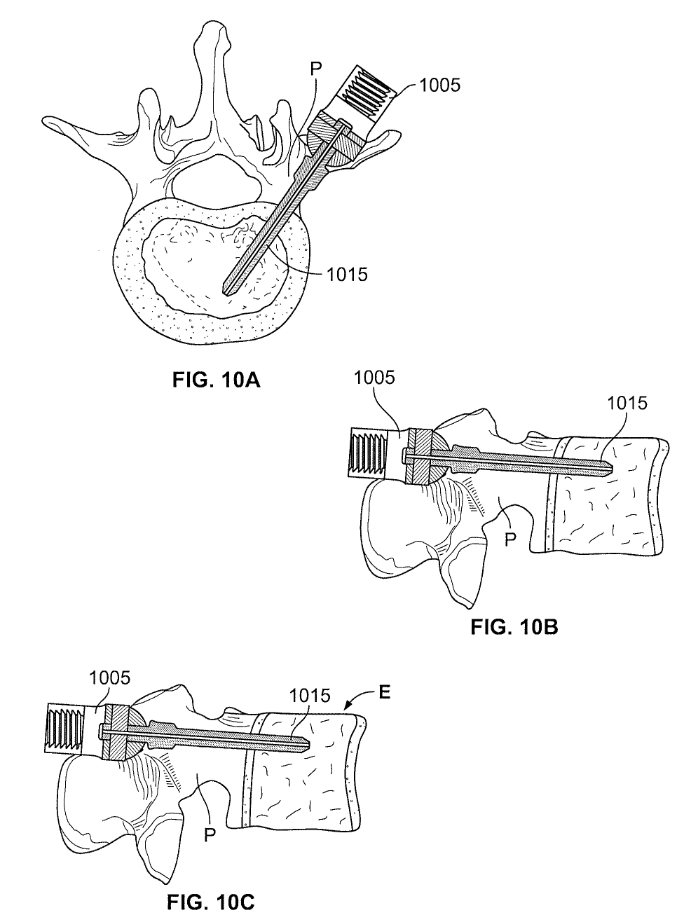

As mentioned, an implant 1005 can be implanted into at least a portion of a vertebra such that the shank 1015 extends into and/or through one or more vertebrae (see FIGS. 10A-10C). In an embodiment, the implant 1005 is positioned into the pedicle, although it can be positioned into any region of the vertebra. Moreover, the implant 1005 can be a threaded screw or it can be any other type of implant whether threaded or non-threaded. FIG. 10B illustrates an embodiment in which the implant 1005 is positioned into or adjacent to the intervertebral disc space. In another embodiment, the implant 1005 is positioned into or adjacent to the vertebral endplate E (see FIG. 10C). The implant 1005 can penetrate the endplate E such that a distal end of the implant 1005 extends into the disc space. In another embodiment, the implant 1005 is implanted into the pelvic wall.

The disclosed devices, treatments and methods can be used to treat spinal deformities such as idiopathic adolescent scoliosis, as follows. Implants are positioned, as described above, into vertebrae on the concave (inner) side of the curve as well as the convex (outer) side of the curve. Through the implant on the concave side of the curve, growth-stimulating treatments can be delivered. The implant on the concave side can be connected to catheters that are then used to deliver growth stimulating factors, such as for example insulin-like growth factor types I or II. Simultaneously, the implant on the convex side of the curve can be connected to catheters that are used to deliver growth inhibitory factors, such as for example insulin like growth factor binding protein, type I.

As described above the implant and associated delivery catheters can be connected to reservoirs, access ports, and/or pumps or any combination thereof. The localized and selective treatment protocol retards growth on the convex side of the curve while stimulating the concave side to grow or ‘catch up’ to the convex side of the curve therein straightening the vertebrae.

Alternatively, fixation assemblies can be implanted in the vertebrae on the convex side of the curve. The fixation assemblies can be joined by a connecting element such as a longitudinal rod or rods to fix the outer, convex side of the spine and keep the curve from progressing while the concave side of the curve can continue growing.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A bone therapy method, comprising:
providing an implant, the implant comprising an internal passageway with a proximal opening and a distal opening;
positioning the implant into a vertebra such that the distal opening is adjacent a delivery site, wherein the delivery site comprises an intervertebral disc space or a vertebral endplate; and
delivering a therapeutic substance through the proximal opening and through the internal passageway to the delivery site while the implant is positioned in the vertebra.

2. A method as in claim 1, wherein the therapeutic substance comprises at least one of a cellular element, cell therapy agent, radiographic material, osteoproliferative material, osteogenic material, osteoinductive material, chondroproliferative factor, chondrogenic factor, extracellular matrix-inducing factor, antibiotic, analgesic, anesthetic, anti-inflammatory agent, therapeutic protein, cytokine, and chemokine.

3. A method as in claim 1, wherein the therapeutic substance comprises at least one of an insulin-like growth factor type I, insulin-like growth factor type II and insulin-like growth factor binding protein-type 1.

4. A method as in claim 1, wherein positioning the implant into a vertebra comprises positioning the implant into a pedicle of the vertebra.

5. A method as in claim 1, wherein the implant is a bone screw

6. A method as in claim 5, wherein the bone screw is a monoplanar, polyaxial bone screw.

7. A method as in claim 1, wherein the internal passageway extends through the implant and wherein the distal opening is at a distal tip of the implant.

8. A method as in claim 1, further comprising inserting a delivery catheter into the internal passageway, wherein the therapeutic substance is delivered via the delivery catheter.

9. A method as in claim 8, further comprising providing at least one reservoir, wherein the reservoir contains the therapeutic substance delivered via the delivery catheter into the internal passageway of the implant.

10. A method as in claim 9, wherein the reservoir is located subcutaneously or subfascially.

11. A method as in claim 9, wherein the reservoir comprises an external needle access port.

12. A method as in claim 11, wherein the reservoir is affixed to an ilium bone.

13. A method as in claim 1, further comprising programming a pump, the pump controlling the delivery of the therapeutic substance to the delivery site.

14. A method as in claim 5, further comprising connecting the bone screw to a fixation rod.

15. A method as in claim 14, wherein the fixation rod comprises fenestrations.

16. A method of treating a patient having idiopathic adolescent scoliosis, comprising:
providing a first implant, the first implant comprising an internal passageway with a proximal opening and a distal opening;
providing a second implant, the second implant comprising an internal passageway with a proximal opening and a distal opening;
positioning the first implant into a vertebra on a concave side of the patient's spine such that the distal opening of the first implant is adjacent a first delivery site;
positioning the second implant into a vertebra on a convex side of the patient's spine such that the distal opening of the second implant is adjacent a second delivery site;
delivering a growth-stimulating agent to the first delivery site through the first implant to stimulate growth on the concave side of the spine; and
delivering a growth-inhibitory agent to the second delivery site through the second implant to retard growth on the convex side of the spine.

17. A method as in claim 16, wherein the growth-stimulating agent comprises at least one of insulin-like growth factor type I and insulin-like growth factor type II.

18. A method as in claim 16, wherein the growth-inhibitory agent comprises insulin-like growth factor binding protein, type I.

19. A method as in claim 16, wherein at least one of the growth-stimulating agent and growth-inhibitory agent is delivered through a catheter that is positioned in a respective first or second implant.

20. A method as in claim 16, further comprising connecting the first or second implant to at least one of a reservoir, access port, and pump.
